(19) United States
(12) Patent Application Publication
(10) Pub. No.: US 2026/0226555 A1
(43) Pub. Date: Aug. 6, 2026

(54) AMPLICON-BASED APPROACH FOR DETECTING DIFFERENCES IN HUMAN DNA FRAGMENTATION PATTERNS BETWEEN CANCER AND NON-CANCER SAMPLES

(71) Applicant: THE TRANSLATIONAL GENOMICS RESEARCH INSTITUTE, Phoenix, AZ (US)

(72) Inventors: Cristian Tomasetti, San Juan Capistrano, CA (US); Kamel Lahouel, Scottsdale, AZ (US); Stephanie J. Kosakovsky Pond, Gladwyne, PA (US); Jeffrey Trent, Paradise Valley, AZ (US); Candice L. Wike, Glendale, AZ (US); Victoria L. Zismann, Chandler, AZ (US); Kameron Bates, Phoenix, AZ (US)

(21) Appl. No.: 19/154,814

(22) PCT Filed: Feb. 9, 2024
(86) PCT No.: PCT/US2024/015236

§ 371 Date: Aug. 8, 2025

Related U.S. Application Data
(60) Provisional application No. 63/484,146, filed on Feb. 9, 2023.

(51) Int. Cl. C12Q 1/6886; C12Q 1/48; C12Q 1/6811; C12Q 1/6813; C12Q 1/686; G16B 20/10; G16B 30/00

(52) U.S. Cl. CPC C12Q 1/6886; C12Q 1/48; C12Q 1/6811; C12Q 1/6813; C12Q 1/686; C12Y 207/07; G16B 20/10; G16B 30/00; C12Q 2600/112

(57) ABSTRACT

Disclosed herein is a method of detecting cancer by amplifying two or more regions of interest in the DNA; analyzing the amplified product to determine a length distribution of sequences of the amplified product; and comparing with the analogous length distribution of a control. A statically significant difference in distribution compared to the control DNA sample indicates detection of cancer. A set of primers for each amplicon is used for PCR, preferably the primers used have about 10-25 base pairs motif followed by 4-8 base pair kmer in the 5′ to 3 direction and 4-8 base pair kmer in the 3 to 5′ direction followed by an about 10-25 bp motif. The amplified product here often comprises a first amplicon with an average length of 35 to 45 base pairs and a second amplicon with an average length of 55 to 65 base pairs between the 5′ to 3′ and 3′ to 5′ primers.

### CROSS-REFERENCE TO RELATED APPLICATIONS

This application claims priority to U.S. Provisional Patent Application No. 63/484,146, filed Feb. 9, 2023, the contents of which are incorporated herein by reference in its entirety.

### TECHNICAL FIELD

This document relates to an amplicon-based approach for detecting differences in human DNA fragmentation patterns between cancer and non-cancer samples.

### REFERENCE TO SEQUENCE LISTING SUBMITTED ELECTRONICALLY

The official copy of the sequence listing is submitted electronically via Patent Center as ST.26 XML format having the file name “91482_262WO-PCT.xml” created on Feb. 9, 2024, and having a size of 53,884 kilobytes, and is filed concurrently with the specification. The Sequence Listing ST.26 XML file is part of the specification and is herein incorporated by reference in its entirety.

### BACKGROUND

Cancer can be caused by inherited and environmental factors as well as by changes to DNA resulting from random, unpredictable DNA copying errors. Early detection of cancer can often be an important factor in effective treatment. DNA sequences are known to differ between DNA obtained from cancer cells versus normal cells. See, for example, WO2020236625.

“Liquid biopsy” has recently emerged as a diagnostic modality in human medicine, particularly cancer medicine. It consists of fluid-based genomic profiling that can be performed by analyzing circulating free DNA (cfDNA). cfDNA is released into the blood and other body fluids after cell death by necrosis and apoptosis, or by active secretion. Since it is a non-invasive approach, in can be repeated many times, with little or no discomfort from the patient. When a subject has cancer, a portion of the cfDNA may be cancer-derived, which defined as circulating tumor DNA (ctDNA).

There is a need for better, cheaper and more efficient screening techniques to determine if an individual has cancer.

### SUMMARY

Some aspects comprise 1. A method of detecting cancer comprising: providing a human DNA sample from at least one subject suspected of having cancer; amplifying two or more regions of interest in the human DNA using polymerase chain reaction (PCR) to produce amplified products; analyzing the amplified products to determine a length distribution of sequences of the amplified products; and comparing the length distribution of the amplified products with an analogous length distribution of a non-cancerous human DNA sample; wherein a statically significant difference in distribution of the subject suspected of having cancer versus the distribution of the non-cancerous human DNA sample indicates the likely presence of cancer; wherein the PCR is performed using a set of PCR primers for each region of interest, the primers comprising a forward primer comprising an about 10-25 base pairs first sequencing primer followed a first 4-8 base pair kmer in the 5′ to 3′ direction and (ii) a reverse primer comprising 4-8 base pair second kmer followed by an about 10-25 bp second sequencing primer in the 3′ to 5′ direction, and wherein the amplified products comprise a first amplicon with an average length of 35 to 45 base pairs and a second amplicon with an average length of 55 to 65 base pairs between the 5′ to 3′ and 3′ to 5′ primers.

In some aspects, the first amplicon has an average length of about 40 base pairs and the second amplicon has an average length of about 60 base pairs between the 5′ to 3′ and 3′ to 5′ primers.

In other aspects, the first amplicon has an average total length of 47 to 52 base pairs and the second amplicon has an average total length of 67 to 72 base pairs.

In one aspect, the first amplicon and the second amplicon are produced by two different primers. In another aspect, the first amplicon and the second amplicon are produced by a primer pair or a three primer configuration comprising two forward and one reverse primer.

In some embodiments, the primers comprise (i) an about 10-25 base pair first sequencing primer followed by 5-7 base pair first kmer in the 5′ to 3′ direction and (ii) a 5-7 base pair second kmer in the 3′ to 5′ direction followed by an about 10-25 bp second sequencing primer. In one embodiment, one of the first and second kmers comprises 5 base pairs and the other kmer comprises 7 base pairs.

In certain aspects, the non-cancerous length distribution is obtained from human DNA of one or more non-cancerous subjects.

In some aspects, the disclosed methods further comprise obtaining an aneuploidy score comprising: clustering first and second amplicon counts into a short mode and a long mode respectively; normalizing each count by dividing each amplicon count by the total number of amplicon counts coming from the corresponding mode and the corresponding chromosome arm; randomly sampling a first number of amplicons from each mode; generating a first score for every sample and mode within the first number of amplicons and performing a first gene set variation analysis (GSVA); randomly sampling a second number of amplicons from each mode; generating a second score for every sample and mode within the second number of amplicons and perform a second gene set variation analysis (GSVA); averaging the scores from the first GSVA and the second GSVA; and determining mode specific aneuploidy scores.

In other aspects, the disclosure provides a method of detecting a cancer in a human patient by analyzing genomic DNA fragmentation, the method comprising: providing a human DNA sample from the human patient; amplifying a plurality of regions of interest in the DNA using polymerase chain reaction (PCR) to produce amplified products; analyzing the amplified products to determine a length distribution of sequences of the amplified products; and comparing the length distribution of the amplified products with an analogous length distribution of a non-cancerous human DNA sample; wherein an increase in the ratio of shorter amplified products to longer amplified products in the length distribution of the DNA sample from the human patient compared to that in the non-cancerous human DNA sample indicates increased genomic DNA fragmentation and identifies the cancer.

In some aspects, the PCR is performed using a set of PCR primers for each region of interest, each set of PCR primers comprises a forward primer with a first 4-8 base pair kmer in the 5′ to 3′ direction and a reverse primer with a 4-8 base pair second kmer in the 3′ to 5′ direction, the first kmer and second kmer in a set of PCR primers are selected to amplify genomic DNA from the human patient resulting in a population of amplicon lengths with a mode characteristic of each region of interest, the modes characteristic of the plurality of the regions of interest are determined from the amplified products, and the difference in length between modes is at least 10 bp, at least 15 bp, at least 20 bp, at least 25 bp, or at least 30 bp.

In other aspects, the plurality of modes comprises a first mode of about 35 bp to about 55 bp and a second mode of about 55 bp to about 75 bp.

In yet other embodiments, the disclosure provides a method of detecting a cancer in a human patient by analyzing motif distributions, the method comprising: providing a human DNA sample from the human patient; amplifying a plurality of regions of interest in the DNA using polymerase chain reaction (PCR) to produce amplified products wherein the amplified products comprise a plurality of motifs; mapping each motif to a genomic region from the human DNA sample; determining a probability distribution for each motif to generate a profile of motif distributions for the human DNA sample; and comparing the profile of motif distributions for the human DNA sample to an analogous profile of motif distributions from a non-cancerous human DNA sample; wherein significant differences in probability distributions for the human DNA sample and the non-cancerous human DNA sample identifies the cancer.

In certain aspects, analyzing motif distributions comprises analysis with kernel support vector machine (SVM). In one aspect, kernel SVM comprises spectrum representation kernel.

In yet other aspects, the human DNA sample and/or the human non-cancerous DNA sample is cell-free DNA. In one aspect, detecting the cancer further comprises aiding in cancer diagnosis; disease monitoring prior to, during, and/or after treatment; minimal residual disease (MRD) detection; or any combination thereof.

In some aspects, the disclosed methods further comprise administering a cancer therapy to the human patient. In one aspect, the cancer therapy is surgical resection, chemotherapy, radiation therapy, or a combination thereof.

In other embodiments, the disclosure provides a method of detecting aneuploidy comprising: providing a human DNA sample from at least one subject suspected of having cancer; amplifying two or more regions of interest in the human DNA using polymerase chain reaction (PCR) to produce amplified products; analyzing the amplified products to determine an amplicon count for each amplified product length; clustering the first and second amplicon counts into a short mode and a long mode respectively; normalizing each count by dividing each amplicon count by the total number of amplicon counts and the corresponding chromosome arm; randomly sampling a first number of amplicons from each mode; generating a first score for every sample and mode within the first number of amplicons and perform a first gene set variation analysis (GSVA); randomly sample a second number of amplicons from each mode; generating a second score for every sample and mode within the second number of amplicons and perform a second gene set variation analysis (GSVA); averaging the scores from the first GSVA and the second GSVA; and determining mode specific aneuploidy scores; wherein the PCR is performed using a set of PCR primers for each region of interest, the primers comprising a forward primer comprising an about 10-25 base pairs first sequencing primer followed a first 4-8 base pair kmer in the 5′ to 3′ direction and (ii) a reverse primer comprising 4-8 base pair second kmer followed by an about 10-25 bp second sequencing primer in the 3′ to 5′ direction, and wherein the amplified products comprise a first amplicon with an average length of 35 to 45 base pairs and a second amplicon with an average length of 55 to 65 base pairs between the 5′ to 3′ and 3′ to 5′ primers.

In one aspect, the first amplicon and the second amplicon are produced by two different primers. In another aspect, the first amplicon and the second amplicon are produced by one primer.

In some aspects, the first amplicon has an average total length of 47 to 52 base pairs and the second amplicon has an average total length of 67 to 72 base pairs. In other aspects, the first amplicon has an average total length of about 50 base pairs and the second amplicon has an average total length of about 70 base pairs. In other aspects, one of the first and second kmers comprises 5 base pairs and the other kmer comprises 7 base pairs.

In other embodiments, the disclosure provides a method of selecting a primer of the structure SP-kmer wherein SP is sequencing primer and kmer comprises 4-8 base pairs that are commonly positioned immediately to the 5′ or 3′ side of a target sequence in a human DNA sample; the method comprising: determining DNA sequences of a plurality of amplicons within target DNA; determining kmer sequences on the 5′ or 3′ side of the amplicons; constructing a plurality of test primers; contacting target DNA with a plurality of test primers and plotting density of fragment count versus fragment length; selecting a primer that has a single peak of the plot of product count versus product length and has a higher than average density.

Yet other aspects concern primers of the compositions disclosed herein. In certain aspects, the disclosed primers comprise a sequence selected from the group consisting of SEQ ID NOs: 1-60.

The foregoing and other aspects, features, and advantages will be apparent from the DESCRIPTION and DRAWINGS, and from the CLAIMS if any are included.

### BRIEF DESCRIPTION OF THE DRAWINGS

Implementations will hereinafter be described in conjunction with the appended and/or included DRAWINGS, where like designations denote like elements, and:

FIG. 1 is a representation of the size of fragment distribution of free cell DNA obtained from normal cells and cancerous individuals.

FIG. 2 illustrates an embodiment where a method disclosed herein operates by utilizing repeat regions of the genome and designing primers for two PCR amplicons with an expected unique insert size between the repetitive 5mer and 7mer.

FIG. 3 is a representation of an embodiment showing fragment size distribution of cancer versus normal DNA.

FIGS. 4-8 present a representation of generation of the primer and insert combinations.

FIGS. 9 and 10 present data obtained using the instant method to detect cancer in bloodhounds and German Shepherds.

FIGS. 11A and 11B present data showing ratios of two domains for cancer and healthy dogs.

FIG. 12 presents data showing ratios by the instant method from two healthy human samples and two cancer samples where a ratio is computed for each chromosome.

FIG. 13 presents data showing ratios obtained by data generated from the instant method of healthy human samples, gastric, and lung cancer samples.

FIG. 14 presents data showing difference in normalized protection scores obtained by data generated from the instant method of healthy human samples, gastric, and lung cancer samples.

FIGS. 15A-15C present ROC curves summarizing the performance of 414 samples for fragmentation, aneuploidy, and motif distribution markers, respectively.

### DETAILED DESCRIPTION

Detailed aspects and applications of the disclosure are described in the drawings and detailed description of the technology. Unless specifically noted, it is intended that the words and phrases in the specification and the claims be given their plain, ordinary, and accustomed meaning to those of ordinary skill in the applicable arts.

ctDNA has been shown to be a surrogate for tumor tissue DNA because it can carry the same genomic alterations. It has been used alone or in combination with tumor tissue samples to profile cancer patients for research and diagnostic purposes. While tumor tissue testing provides a snapshot in time and space of a cancer's complexity for a specific tumor site, ctDNA analysis is able to more comprehensively capture the heterogeneity of the overall disease throughout the body and across different tumor lesions. Moreover, since tumor DNA can be shed into the bloodstream even when cancer is otherwise undetectable and because ctDNA has distinctive features which make it suitable for discrimination of cancer-bearing patients from healthy subjects, it can also be used to detect cancer early, as a screening tool in the general population.

In human cancer diagnostic research, several efforts have been made to develop a liquid biopsy test to be used as a multi-cancer early detection tool to identify cancer in the asymptomatic population when cancer can be eradicated surgically. If sufficiently specific and sensitive, such a test could play a major role in improving overall cancer survival and reducing long-term costs of care.

A need exists for a liquid biopsy test to be used as a multi-cancer early detection tool to identify cancer in the asymptomatic population when cancer can be eradicated surgically. A sufficiently specific and sensitive test could play a major role in improving overall cancer survival and reducing long-term costs of care.

This disclosed methods, including assays and analysis methods, utilize repetitive DNA elements to identify a bi-modal distribution of ctDNA fragments to facilitate cancer versus non-cancer discrimination. The methods include a genomic fragment-based (fragmentomics) circulating tumor (ctDNA) analysis from blood. The disclosed methods can be used in cancer detection, to aid in cancer diagnosis, for disease monitoring prior to, during, and after treatment, and in minimal residual disease (MRD) detection.

The detection of MRD can play a significant role not only in monitoring a patient's response to therapy, but also in the accurate diagnosis of the underlying cause of major clinical signs. MRD typically refers to the presence of malignant cells (usually in reference to leukemic cells) that are not detectable on the basis of cellular morphology. Several studies have shown that quantitative detection of MRD in lymphoid malignancies predicts clinical outcome. (Szczepanski T, et al., Lancet Oncol 2001; 2:409-17; van Dongen J J, et al., Lancet 1998; 352:1731-8; Bruggemann M, et al., Acta Haematol 2004; 112:111-9; Cave H, et al., N Engl J Med 1998; 339:591-8; Coustan-Smith E, et al., Blood 2000; 96:2691-6; Coustan-Smith E, et al., Blood 2002; 100:52-8; Wells D A, et al., Am J Clin Pathol 1998; 110:84-94; Radich J, et al., Biol Blood Marrow Transplant 1995; 1:24-31; Bahloul M, et al., Best Pract Res Clin Haematol 2005; 18:97-111; Hoshino A, et al., Tohoku J Exp Med. 2004; 203:155-64; Ciudad J, et al., Br J Haematol 1999; 104:695-705; Lucio P, et al., Leukemia 1999; 13:419-27.)

Monitoring the response of a cancer patient to a therapeutic treatment on the basis of tumor load quantification (e.g., by MRD detection) may assist in the assessment of a relative risk of relapse and can also be used to identify patients who may benefit from therapy reduction, therapy intensification, reduction of immunosuppression for graft-versus-leukemia effect after a stem cell transplant, or adoptive T cell therapy. (Bradfield S M, et al., Leukemia 2004; 18:1156-8.) Minimal disease may also be encountered in diagnostic situations. For example, low levels of monoclonal B cells in patients presenting clinically with cytopenia may raise suspicions for a diagnosis of myelodysplastic syndrome. (Wells et al., Blood 2003; 102:394-403.) Minimal disease detection is also encountered in staging of lymphoma, which may involve the detection of low levels of tumor cells against a background of normal cells. The detection of minimal disease as described herein (e.g., as MRD detection in lymphoid cancer patients following treatment) need not be limited to monitoring the effects of treatment but may also find uses in diagnostic settings.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

The term “plurality”, as used herein, means more than one.

“Gene set variation analysis” (GSVA) is a technique known in the art that uses a single sample to provide an estimate of variation within a gene set.

“Aneuploidy” is a score that is calculated as the sum of the altered arms of chromosomes and is a measure of chromosomal abnormalities.

The term “kmer” refers to a short base pair sequence where the k represents the number of base pairs in the sequence. For example, a 5mer has 5 base pairs and a 7mer has 7 base pairs.

“Primer” refers to a short segment of synthesized DNA that target unique sequences in a target DNA sample.

The polynucleic acid produced by the amplification technology employed is generically referred to as an “amplicon” or “amplification product.”

The disclosure concerns an amplicon-based approach for detecting differences in fragmentation patterns between cancer and non-cancer samples. Non-cancer samples are also referred to herein as normal samples. The fragmentation patterns are found to differ between cancer and normal cells in part due to the fact that specific genomic regions are more protected or less disrupted in normal individuals (or individuals without cancer) than in individuals with cancer.

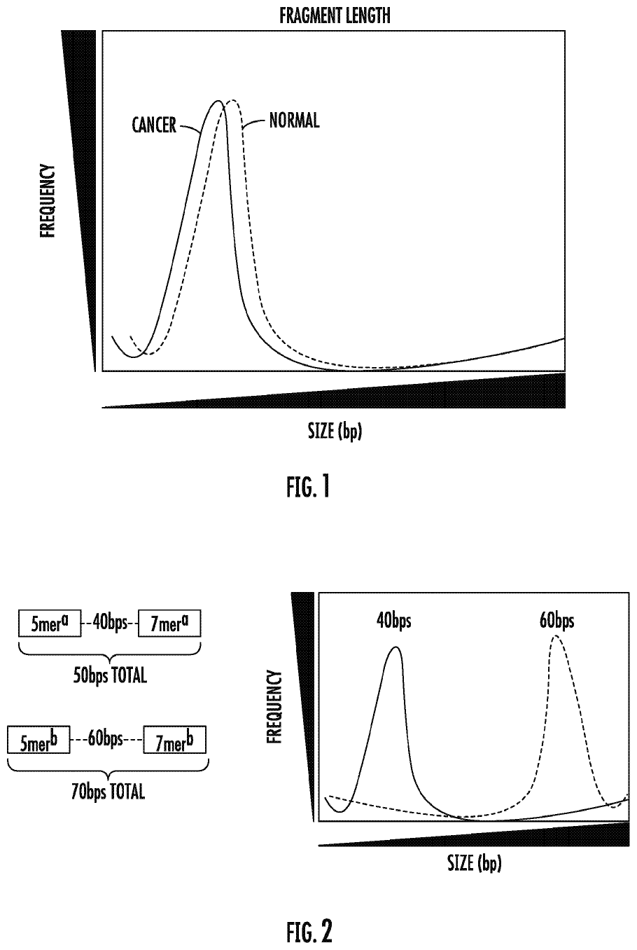

The distribution in FIG. 1 presents a representative size of the fragment from shallow sequencing of cancer and non-cancer DNA. In some cases, there can be a small distinction between non-cancer (“normal”) and cancer distributions.

One important facet of the instant methodology is that the probability that a region is affected by fragmentation must decrease exponentially with its length. The rapidity of this exponential decrease is governed by the intensity of fragmentation in the considered region. Based on this fact, a second important element of the disclosed methodology is that in order to better detect these differences in fragmentation intensity of cell-free DNA—between cancer and healthy individuals—it can be important to have as many amplicons of (at least two) sufficiently different lengths represented as possible. Typically, cell free DNA (cfDNA) is about 150 base pairs (bp) long which limits the lengths of the two or more fragments.

Polymerase Chain Reaction (PCR) uses a short synthetic sequence or fragment (called a primer) to select a segment of DNA to be amplified. The resulting product of amplification is called an amplicon. In some aspects, as illustrated in FIG. 2, the methods disclosed herein operate by utilizing repeat regions of the genome and designing two or more PCR primers to capture unique insert sequences between the repetitive kmers (about 5mer and about 7mer, in some embodiments). The two primer sets will bind to many locations across the genome and create an average amplicon size of about 50 and about 70 base pairs in some embodiments. A resulting bimodal distribution of amplicons presents differently in cancer-free patients (“normal” patients) vs cancer patients.

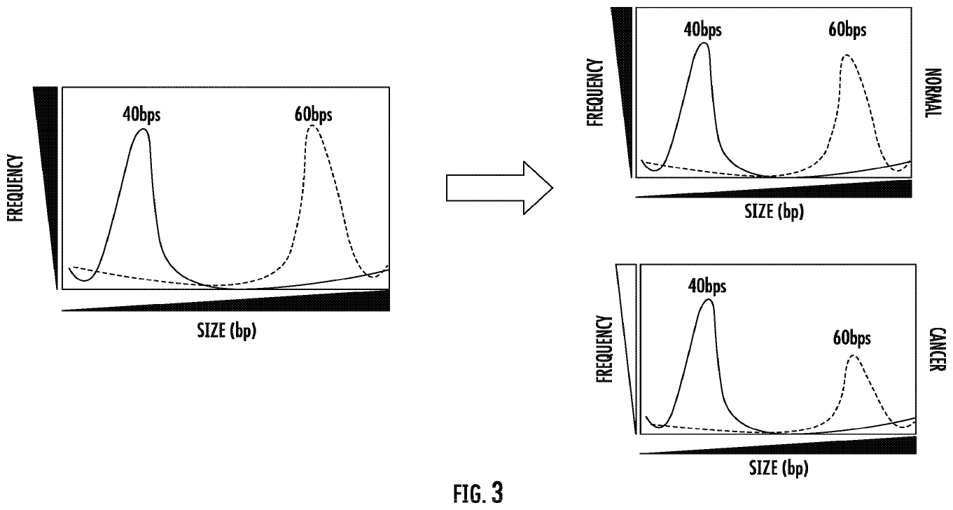

As illustrated in FIG. 3, the disclosed method works by utilizing repeat regions of the genome and designing two or more primers to capture unique insert sequences between the repetitive 5mer and 7mer to produce PCR amplicons. The two primer sets will bind to many locations across the genome and create an average amplicon size about 50 and about 70 base pairs in some embodiments.

The optimal theoretical amplicons length distribution is a distribution that is concentrated around two different lengths and these two lengths should be as different as possible. However, one is limited by many factors such as amplification efficiency, the rate of unique occurrences of inserts in the genome and genomic contamination. More precisely, when the distribution of inserts has lengths that are too far apart, the discrepancy in their respective PCR amplification efficiencies increases. Also, amplicons with long inserts are more vulnerable to noise coming from genomic contamination.

In addition, when an insert size is too short, the likelihood of the insert occurring many times in the genome increases. For these reasons, in some embodiments, we choose a target bimodal distribution having two modes about 50 base pairs (bp) and about 70 bp characterizing the size of the insert (even if other modes could be selected). In some embodiments the two modes can be (i) from 40 to 60 base pairs and (ii) 61 to 80 base pairs. In other embodiments, the two modes can be (i) from 45 to 55 base pairs and (ii) 65 to 75 base pairs. In one embodiment, the two modes have a difference in bp lengths of about 10 bp, about 15 bp, about 20 bp, about 25 bp, or about 30 bp. In another embodiment, the difference between the two modes is between 10 bp and 30 bp or between 15 bp and 25 bp.

To achieve this goal, two different approaches were tested. The first approach utilizes two different primer pairs where each of them yields a unimodal distribution. The second approach utilizes a single primer pair that generates a bimodal distribution by itself. Either procedure may be utilized. Where one approach is discussed in the specification, the other approach may be used in analogous way. We describe in this document the procedure implemented to select the candidate primers.

Whether we use two primer pairs or a single primer pair, each primer pair is characterized by an about 10-25 bp motif followed by a kmer (5′ to 3′ direction) for the forward primer and a kmer followed by an about 10-25 bp motif for the reverse primer. The two 10-25 bp motifs may be 10-20, 12-18, 14-16, or 15 bp. For the size of the kmer, we tested the following forward/reverse combinations: 4/6, 6/4, 5/7, 7/5, 6/8, 8/6 base pairs. We did not consider larger size combinations of kmers because we observed that the candidate primers were all captured by the 5/7 combination. For this reason, we describe how we generate the primers using 5mers/7mers combinations.

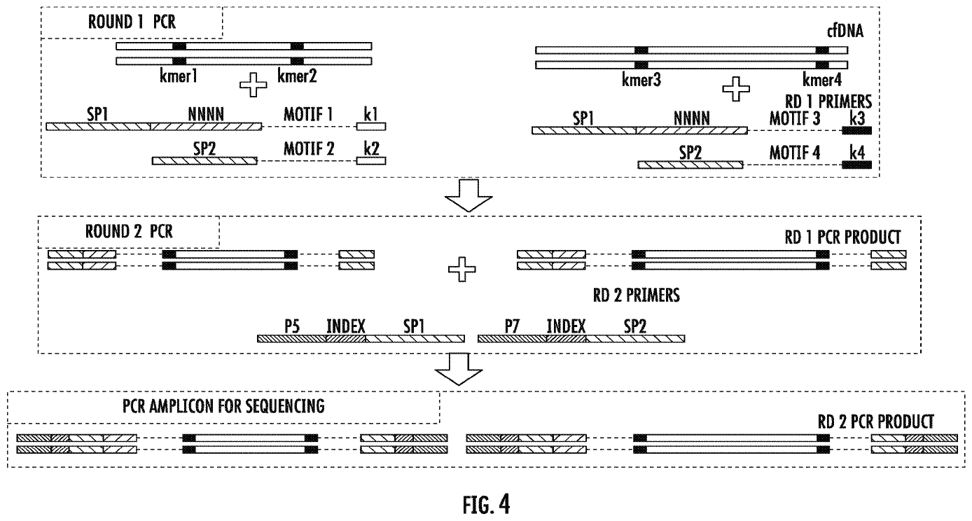

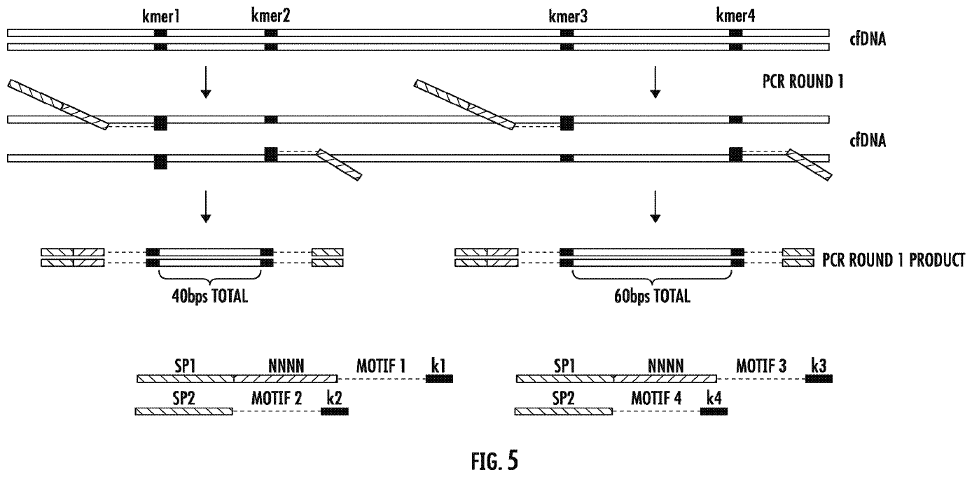

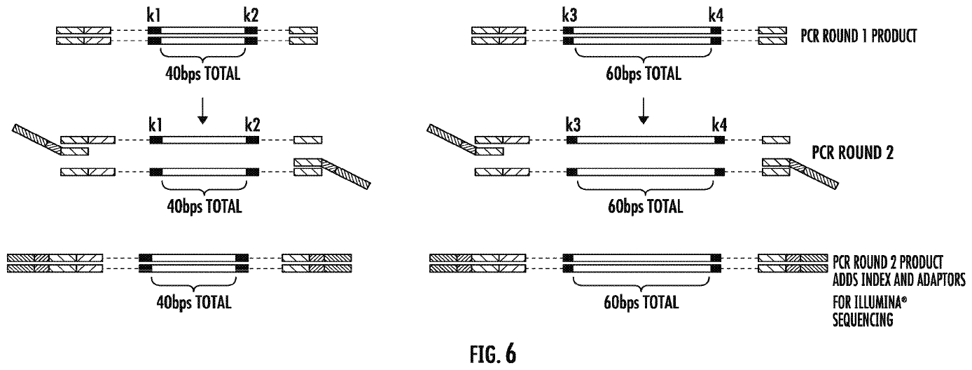

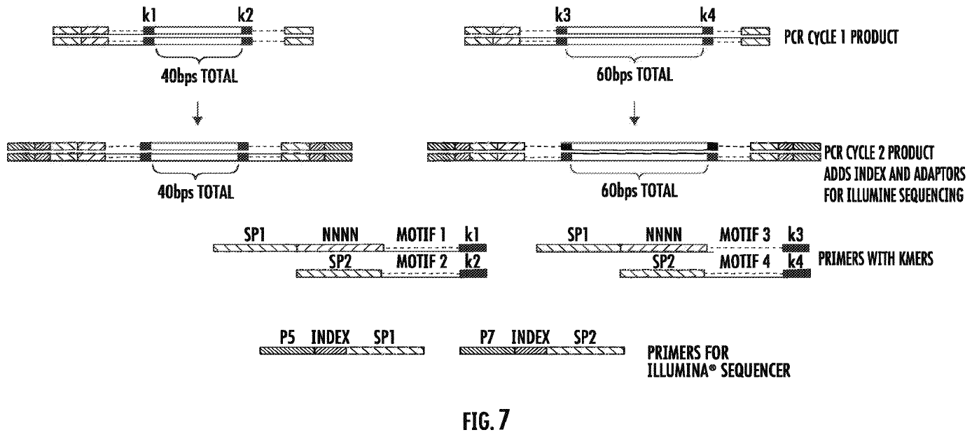

Primer design is illustrated in FIGS. 4-8. Depicted are kmers (k1, k2), motifs (motif 1, motif 2), sequencing primers (SP1 and SP2), ILLUMINA® DNA sequences required for the amplicons to hybridize to the flowcells for next-generation sequencing (P5 and P7) (the disclosed methods can be run on many other next generation sequencing platforms by changing the P5/P7 sequences to other system sequences), series of degenerative nucleotides (NNNN), i.e., unique molecular identifiers (UMIs), and indexes (a series of specific sequences that allow for multiplexing of patient samples in one flow cell acting as barcodes for example). Different versions of the same primers can be possibly constructed by adding different UMIs or motifs sizes (e.g., shorter or longer by one nucleotide), to generate more color diversity per sequencing cycle to improve sequencing accuracy. SP1 and SP2 and other components can be adjusted and maximized for individual applications. FIG. 4 illustrates rounds 1 and 2 of PCR and generation of a PCR amplicon for sequencing. FIG. 5 illustrates PCR round 1 reaction and products from reaction with cfDNA. FIG. 6 illustrates use of indexes and adaptors for next-generation sequencing. The specific sequences at the ends of the amplicons can be varied to be compatible with different next-generation sequencing company flowcells, but sequences from Illumina are used for demonstration. FIG. 7 illustrates PCR round 1 product, PCR round 2 product, primers with kmers, and primers for next-generation sequencing.

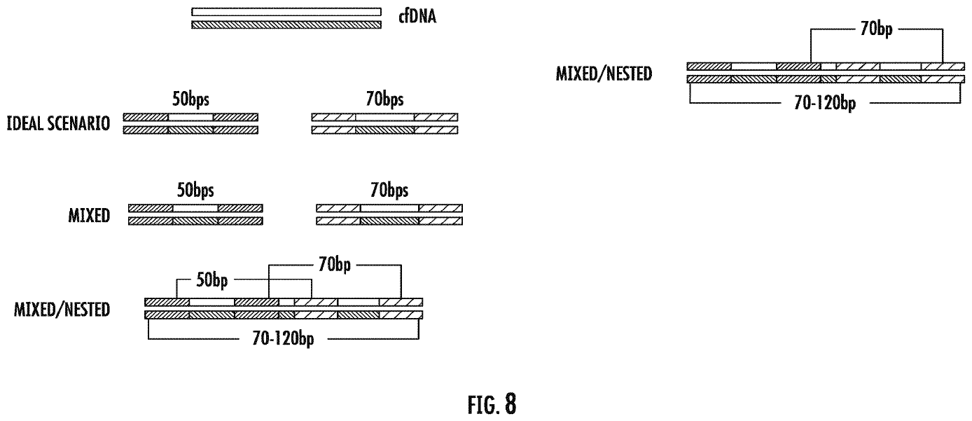

In FIG. 8, the ideal scenario product that is not mixed or nested is compared with mixed and mixed/nested products that can be produced.

In construction of the primers, sets of a certain kmer combinations (5 and 7 or 4 and 6, for example) are selected as test candidates with a particular base pair segment length (40, 50, 60, etc. base pairs) between the kmers. These test candidates are tested 50 times starting with cfDNA position 1 and moving down the strand one base pair at a time. This produces a list of the particular base pair insert with a particular kmer combination. The frequency of counts of a particular base pair length is plotted against the base pair size to find the candidates that produce a bimodal distribution. To achieve this, each primer set needs to produce a substantially single peak. The selected insert should also have commonality between all chromosomes. The actual sequence of the insert, however, may will vary across the chromosomes.

Certain methods use combinations of 5mers/7mers covering the maximum number of unique regions in the genome, such that the distribution of inserts between these kmers is a unimodal about 50 bp, a unimodal about 70 bp or a bimodal with modes at about 50 bp and about 70 bp. We start by looking at the frequency of inserts having a length of 50 plus or minus 5 bp. For each combination of kmers, we calculate the frequency of kmers with a length within that range together with the percentage of obtained inserts that are unique in the genome. We use a frequency of inserts to be larger than 2500 in chromosome 1 and larger than 2500 multiplied by a factor that is equal to the ratio of the chromosome size to the size of chromosome 1 for the other chromosomes. Moreover, we utilize the unique insert rates obtained by the pair to be larger than 90%. Finally, for every kmer pair, we assess the concentration of the insert length distribution within the 45 to 55 bp range. For that, we compute the entropy of the obtained length distribution within that length for every chromosome. We require the entropy of that distribution to be less than 0.99. We then repeat the same procedure with the same constraints but focusing on 70 bp by considering the range 60 to 75 bp.

After this step, we obtain two lists of potential kmer pairs: One for the range centered at 50 bp and the other obtained when considering lengths of about 70 bp. We generate 3 lists from these two lists: One list containing pairs that appeared only in the 50 bp list, one list of kmer pairs that were present only in the 70 bp list and a last one containing pairs appearing on both lists. The first list corresponds to candidates for a primer pair generating a unimodal at 50 bp, the second to candidates for a primer pair that has a unimodal distribution at 70 bp, and the third list pairs providing a bimodal distribution around 50 and 70 bp.

Following this split, for each of the surviving candidates, we generated the full-length distributions, i.e., without restricting to a range of lengths centered around 50 or 70 bp. For each of the pairs of the two first lists, if we observed a new peak, the pair is discarded. For the third list, we explored tri-modal distributions and allowed for at most one additional mode appearing when considering the full range distribution.

The set of pairs surviving the above selection was filtered further by considering cases where the pairs were the same up to a shift or shared one of the kmers. In that case, we kept the pair that has the highest frequency and the most concentrated distribution around the desired mode(s).

Once we obtained a short list of kmer pairs, we constructed the 10-25 bp motifs upstream of the 5mer for the forward primer and downstream of the 7mer for the reverse primer. The forward motif is generated such that it is the most common sequence of length 10-25 upstream of the occurrences of 5mer+inserts+7mer for each 5mer/7mer pair where the sequence is constrained by a resulting melting temperature for the forward primer between 560 and 590 and a GC base pair content between 40% and 60%. The same optimization is applied to generate the 10-25 bp motif downstream of the corresponding 7mer. In the case where the motifs were generated for kmer pairs yielding a bimodal distribution, we observed motifs heavily favoring one mode over the other. In that case we generated an additional motif that optimizes the frequency around inserts coming from the mode that was disregarded by the first motifs. This procedure therefore resulted in kmers that can be attached to two different motifs.

All of the above provided us with a list of primer pairs giving a unimodal distribution centered at a short mode, or a unimodal distribution centered at a long mode, or a bimodal distribution. We considered all combination of short unimodal primer pairs with long unimodal primer pairs. In contrast, the bimodal primer pairs were considered by themselves.

To test these candidate primer pair(s), we use sheared genomic DNA and checked if the obtained distribution from the amplification are close to the predicted bimodal distribution. Primer pair(s) yielding a primer distribution that was not bimodal or was not different from the no-template control (NTC) were discarded.

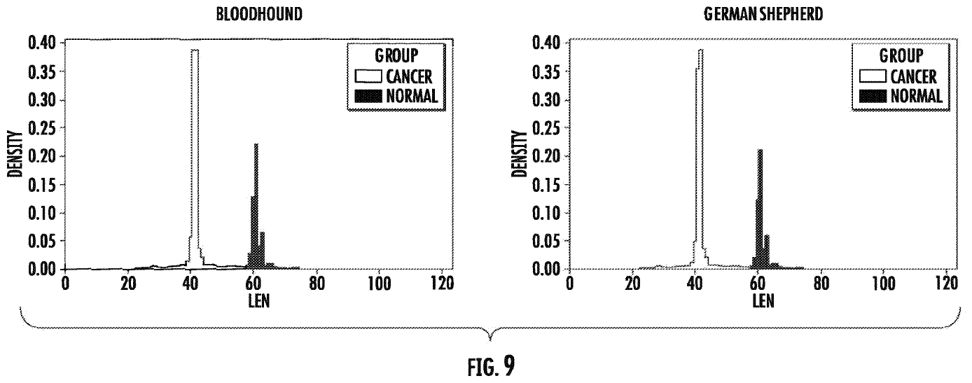

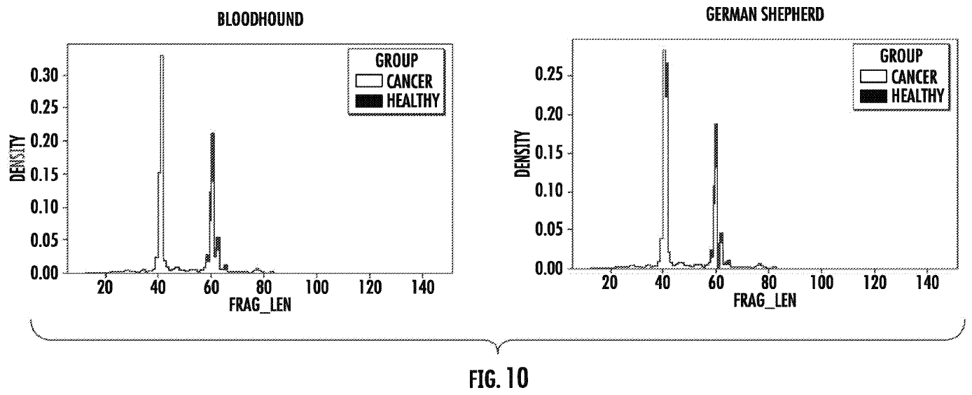

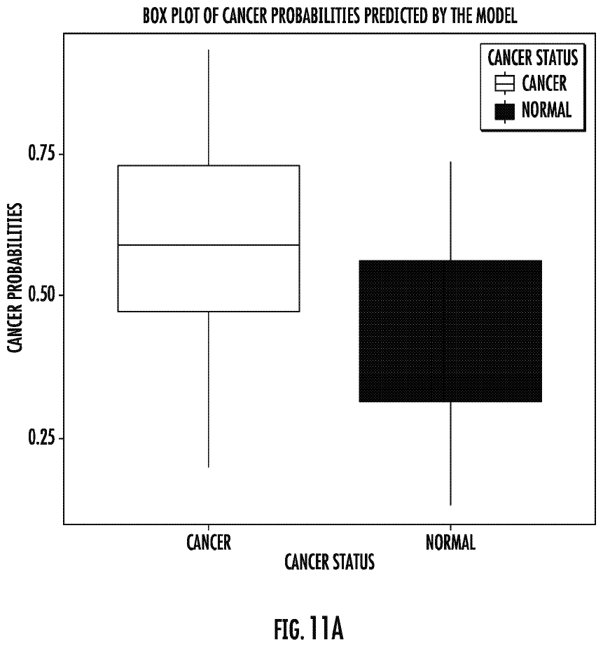

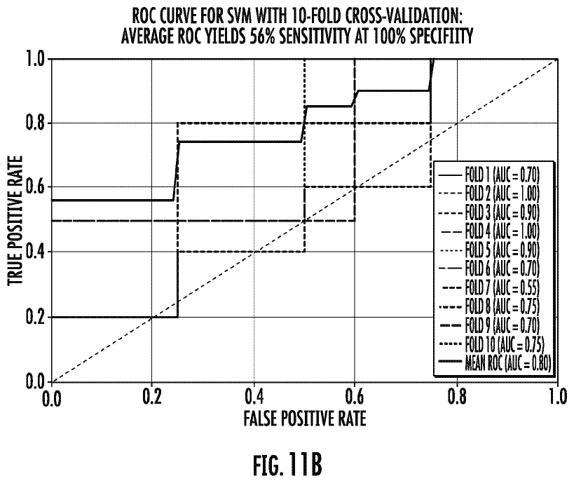

FIGS. 9 and 10 present results from bloodhounds and German Shepherds. In these figures distributions of fragment lengths are illustrated for cancerous DNA and non-cancerous DNA (also referred to herein as “normal DNA”) demonstrating a different fragment length pattern in the caner and normal samples. FIG. 11A plots the cancer probabilities based on the ratio of short- to long-amplicon counts in a cohort of 91 dogs including 48 cancers of multiple tumor types and 43 normal non-cancer controls. The cancer probability is significantly higher in cancer-bearing dogs and allows for the identification of a likely cancer-bearing subject. Sensitivity for multi-cancer detection was 56% and specificity was 100% in this cohort. FIG. 11B plots the average ROC curve generated by the cancer probabilities in FIG. 11A using 10-fold cross-validation.

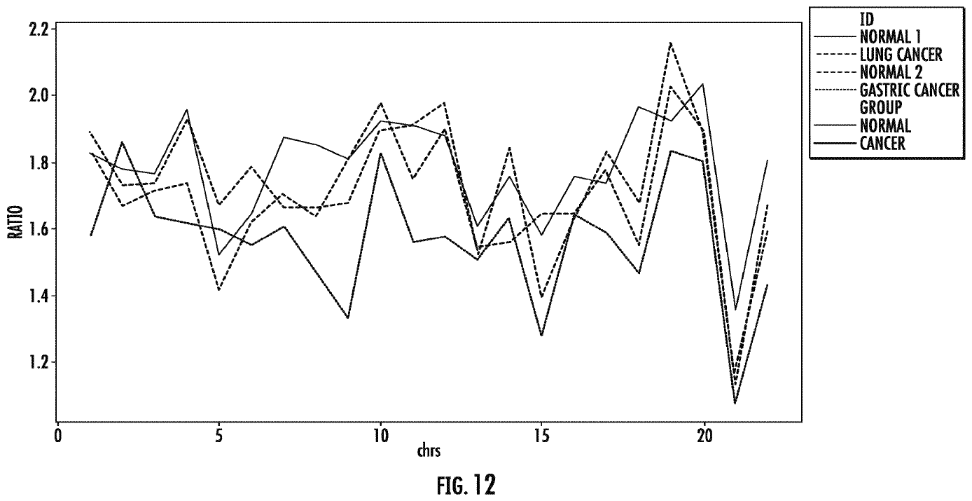

FIG. 12 shows ratios obtained from two healthy human samples and two cancer samples where a ratio is computed for each chromosome. These ratios are lower on average for the cancer samples compared to the healthy ones.

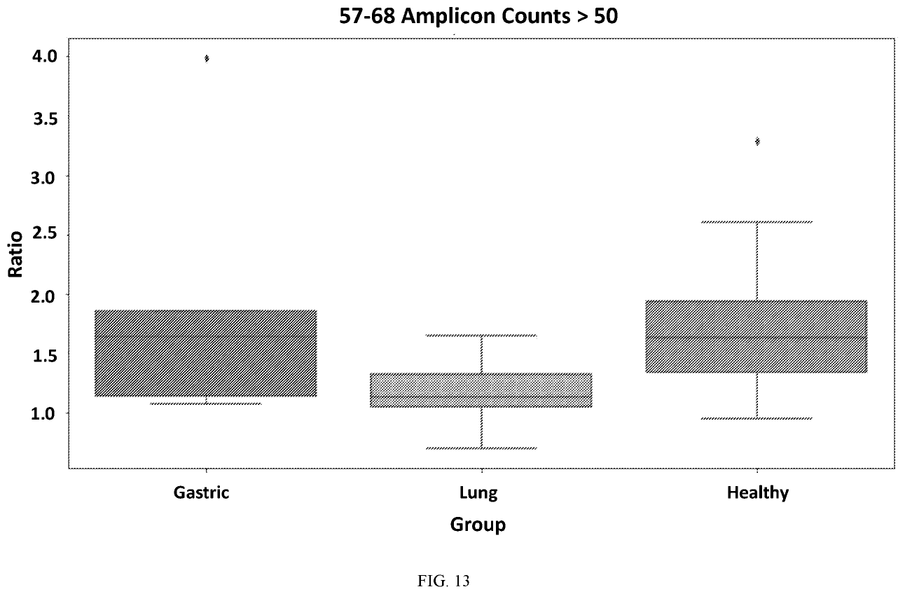

FIG. 13 shows ratios obtained from data of healthy human samples, gastric, and lung cancer samples. These ratios are significantly lower among cancer samples.

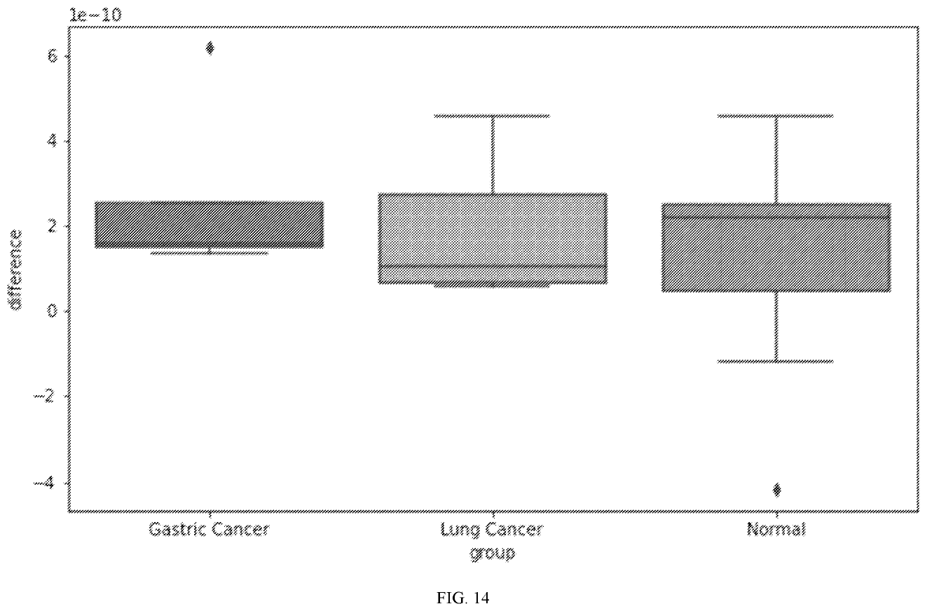

FIG. 14 illustrates difference in normalized protection scores between healthy human samples, gastric, and lung cancer samples. The normalized protection scores are significantly higher in healthy samples.

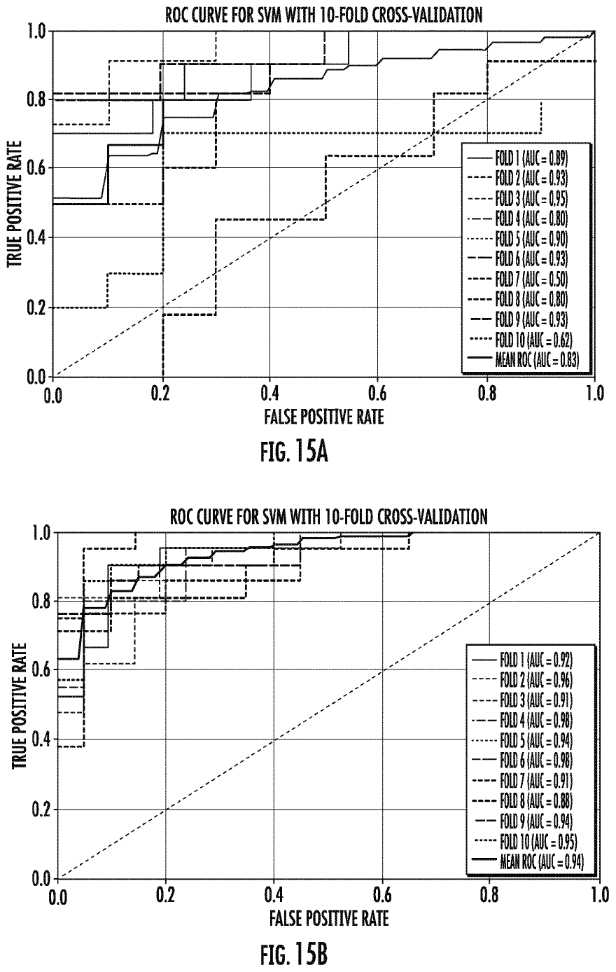

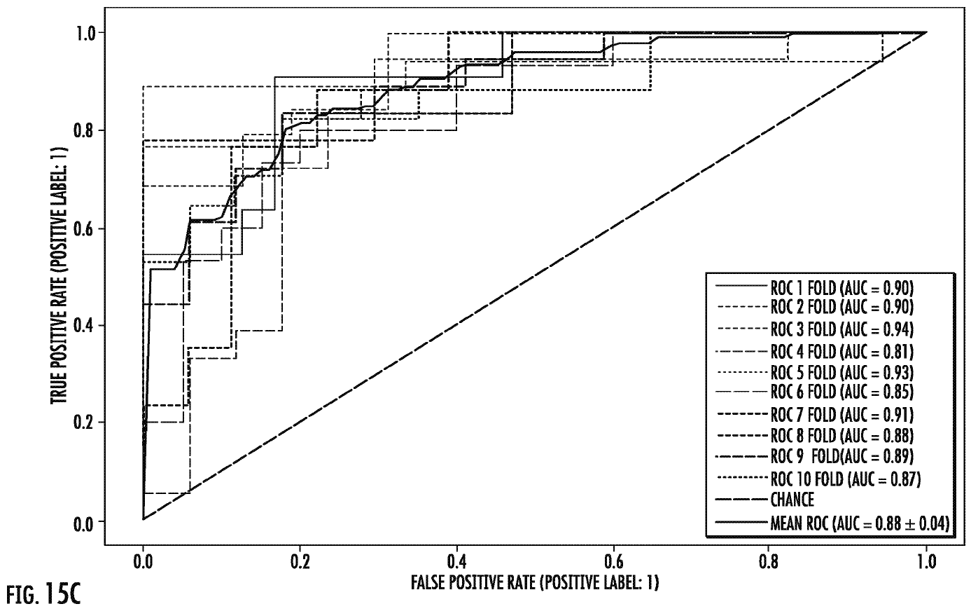

FIGS. 15A-15C present ROC curves summarizing the performance of 414 samples for fragmentation, aneuploidy, and motif distribution markers, respectively.

Table 1 presents representative human primers that can be used with the disclosed methods.

TABLE 1

| Motifs5Mers (FWD) | |
|---|---|
| AGGTTAAAGCCATTCTCCTG | SEQ ID NO: 1 |
| TCTGTAATCCCAGCTACTTG | SEQ ID NO: 2 |
| AATGGTATGATCTCGGCTCA | SEQ ID NO: 3 |
| AAATGGTATGATCTCGGCTC | SEQ ID NO: 4 |
| TTCAAACTAGAGTGCAGTGG | SEQ ID NO: 5 |
| GAGGTAGGAGAATTGCTTGA | SEQ ID NO: 6 |
| AATTAATTAGGCGTGGTGGC | SEQ ID NO: 7 |
| AAAAGCTGAGGCAGGAGAAT | SEQ ID NO: 8 |
| TTCCAGGTTCAAGCGATTCT | SEQ ID NO: 9 |
| TTAACTACTCAGGAGGCTGA | SEQ ID NO: 10 |
| TTTTATTCAGGCTGGAGTGC | SEQ ID NO: 11 |
| AAGTGTAGTGGTGTGATCTC | SEQ ID NO: 12 |
| ATTTTGTTGTCCAGGCTGGA | SEQ ID NO: 13 |
| Motifs7Mers | |
| GCTAATTCCCGGGAATTTTGTAGC | SEQ ID NO: 14 |
| CCACTGCACTCCAACCTAAACG | SEQ ID NO: 15 |
| ACAGGTGTATACCACCACACCC | SEQ ID NO: 16 |
| TTACAGGTATGTGCCACCACGC | SEQ ID NO: 17 |
| ATTCTCCTATCTCAGCCTCCCG | SEQ ID NO: 18 |
| GCACTCCAGTTTGGGTGACAGA | SEQ ID NO: 19 |
| ATCACTTGAGCCCAGGAGTTCG | SEQ ID NO: 20 |
| ACTCCAGTTTGGGTGACAGAGC | SEQ ID NO: 21 |
| GCTAATTCCCGGGATTTTAGTAGC | SEQ ID NO: 22 |
| CCACTGCACTCCAACCTAAACG | SEQ ID NO: 23 |
| AGCCTCCCAAGTAGCTGGGATT | SEQ ID NO: 24 |
| AGTAGCTGGGATTATAGGCGCC | SEQ ID NO: 25 |
| CTCAGCCTCCCAAATAACTGGG | SEQ ID NO: 26 |
| Motifs7Mers | |
| CTGTAGTCCCAGCTACTTAGGAGG | SEQ ID NO: 27 |
| Motifs7Mers (REV) | |
| GCTACAAAATTCCCGGGAATTAGC | SEQ ID NO: 28 |
| CGTTTAGGTTGGAGTGCAGTGG | SEQ ID NO: 29 |
| GGGTGTGGTGGTATACACCTGT | SEQ ID NO: 30 |
| GCGTGGTGGCACATACCTGTAA | SEQ ID NO: 31 |
| CGGGAGGCTGAGATAGGAGAAT | SEQ ID NO: 32 |
| TCTGTCACCCAAACTGGAGTGC | SEQ ID NO: 33 |
| CGAACTCCTGGGCTCAAGTGAT | SEQ ID NO: 34 |
| GCTCTGTCACCCAAACTGGAGT | SEQ ID NO: 35 |
| GCTACTAAAATCCCGGGAATTAGC | SEQ ID NO: 36 |
| CCACTGCACTCCAACCTAAACG | SEQ ID NO: 37 |
| AATCCCAGCTACTTGGGAGGCT | SEQ ID NO: 38 |
| GGCGCCTATAATCCCAGCTACT | SEQ ID NO: 39 |
| CCCAGTTATTTGGGAGGCTGAG | SEQ ID NO: 40 |
| CCTCCTAAGTAGCTGGGACTACAG | SEQ ID NO: 41 |

P5/i5 strand: 5′ (P5 sequence---index----SP1) 3′
P7/i7 strand: 5′ (P7 sequence---index----SP2) 3′

| CTGTAGTCCCAGCTACTTAGGAGG | SEQ ID NO: 42 |
|---|---|
| Full length: | |
| p5/15 strand (5′→3′): | SEQ ID |
| AATGATACGGCGACCACCGAGATCTACACXXXXXXXXACACTCTTTC | NO: 43 |
| CCTACACGACGCTCTTCCGATC*T | |
| p7/17 strand (5′→3′): | SEQ ID |
| CAAGCAGAAGACGGCATACGAGATXXXXXXXXGTGACTGGAGTTCA | NO: 44 |
| GACGTGTGCTCTTCCGATC*T | |
| Motifs5Mers (FWD) | |
| AGGTTAAAGCCATTCTCCTG | SEQ ID NO: 45 |
| TCTGTAATCCCAGCTACTTG | SEQ ID NO: 46 |
| AATGGTATGATCTCGGCTCA | SEQ ID NO: 47 |
| AAATGGTATGATCTCGGCTC | SEQ ID NO: 48 |
| CGCTTTCCCAAAATTAGCCA | SEQ ID NO: 49 |
| TTCAAACTAGAGTGCAGTGG | SEQ ID NO: 50 |
| GAGGTAGGAGAATTGCTTGA | SEQ ID NO: 51 |
| AATTAATTAGGCGTGGTGGC | SEQ ID NO: 52 |
| AAAAGCTGAGGCAGGAGAAT | SEQ ID NO: 53 |
| TTCCAGGTTCAAGCGATTCT | SEQ ID NO: 54 |
| TTAACTACTCAGGAGGCTGA | SEQ ID NO: 55 |
| TTTTATTCAGGCTGGAGTGC | SEQ ID NO: 56 |
| AAGTGTAGTGGTGTGATCTC | SEQ ID NO: 57 |
| ATTTTGTTGTCCAGGCTGG | SEQ ID NO: 58 |

Additional Primers

| | |
|---|---|
| CGACGTAAAACGACGGCCAGTNNNNNNNNNNNNNNNNGGTGA | SEQ ID NO: 59 |
| AACCCCGTCTCTACA | |
| GGTGAAACCCCGTCTCTACA | SEQ ID NO: 60 |

### Features Used to Detect Fragmentation Patterns

The data generated by the amplicon-based approach is defined by amplicon counts for each amplicon and each sample. As described above, the amplicon length distribution is bimodal. Therefore, if we focus on a particular genomic region, one can cluster the amplicon counts into counts coming from short amplicons (e.g., about 50 bp in some embodiments) and counts coming from long amplicons (e.g., about 70 bp in some embodiments). The ratio between the total counts coming from each cluster is a feature indicating the fragmentation intensity in the considered region. We detect change points in these ratios among controls of the training set to partition the genome into regions of uniform fragmentation intensity. To summarize, the features used for classification are the ratios between counts coming from long amplicons to counts coming from short amplicons in each estimated region of uniform fragmentation intensity among the healthy population.

The primer sets amplify certain DNA inserts. The output of the disclosed assay will be the amplicon counts. Amplicon counts mean the number of times the amplicon (amplified DNA inserts) was read across the genomic region. The resulting counts will be in the form of an integer number (0, 1, 2, . . . ), i.e., whole numbers.

The distribution of the lengths of the inserts is bi-modal, thus the distribution of the amplicon counts will be bi-modal. DNA inserts to be amplified are selected to produce this bimodal distribution, and will have one selected long length (for example, approximately 55 bps) and one selected short length (for example, approximately 44 or 45 bps). For each genomic region, the amplicons sizes will be concentrated at these two lengths. A genomic region could be, for example, a single chromosome or a selected number of base pairs. According to various embodiments, a first target insert size, which may also be referred to as the short insert length, may be in the range of 35 to 60 base pairs, or 40 to 60 base pairs, or 45 to 55 base pairs, or 40 to 45 base pairs, or other suitable range. In one embodiment, the first target insert size may be 44 base pairs in length. In another embodiment, the first target insert size may be 45 base pairs in length. In yet another embodiment, the first target insert size may be 38 base pairs in length. According to various embodiments, a second target insert size, which may also be referred to as the long insert length, may be in the range of 40 to 80 base pairs, or 45 to 80 base pairs, or 60 to 80 base pairs, or 61 to 80 base pairs, or 65 to 75 base pairs, or other suitable range. In one embodiment, the second target insert size may be 55 base pairs in length. In another embodiment, the second target insert size may be 64 base pairs in length. In some embodiments, it is preferred that the ranges of the first target insert size and the second target insert size do not overlap. In one example, the first target insert size may be 44 bps or 45 bps and the second target insert size may be 55 bp. In another example, the first target insert size may be 38 bps and the second target insert size may be 64 bp.

Genomic regions of interest can be identified by evaluating sequencing data from one or more healthy patient samples and cancer patient samples. Because some regions of DNA are more fragmented by cancer than other regions, the assay can be used to look at “regions” of interest in the DNA, i.e. areas found to be more affected by fragmentation due to cancer.

A ratio of the count of long amplicons to the count of short amplicons is determined. This ratio (number of long amplicons to short amplicons) is one output that can be used as a cancer diagnostic itself, or this ratio can be further analyzed with additional informatics tools and methods to diagnose the presence or absence of cancer.

In cancer patient samples, the cfDNA tends to be more intensely fragmented than in non-cancerous, normal, or healthy patient cfDNA samples. In terms of the DNA inserts of interest, in a cancer patient both lengths of DNA inserts are affected by cancer and see increased fragmentation. However, the long inserts are more affected by this fragmentation, by nature of their length. In other words, the long inserts are more fragmented by cancer than short inserts. Therefore, when comparing two different DNA insert lengths in a cancer sample compared to a normal sample, a cancer sample tends to have fewer long DNA inserts relative to short DNA inserts. Because the instant assay amplifies a DNA insert only when the entire insert is present and will not amplify if the insert is fragmented (i.e., if the insert is not present as a whole), the count of long amplicons will show a greater decrease in a cancer sample compared to the count of short amplicons in that cancer sample. Lastly, a cancer patient cfDNA sample that is amplified using the primer sets is expected to have a lower ratio of long to short amplicons than a cfDNA sample from a healthy patient.

To determine whether the ratio indicates the presence of cancer, the ratio of long to short amplicons from the sample is compared to a ratio determined from one or more normal (non-cancerous) samples. This normal ratio or control ratio, or even a ratio threshold, can be determined using one or more non-cancer (normal) samples and/or one or more known cancer samples and/or publicly available data. By finding a normal/control ratio based on a set of normal samples and/or data, the statistical probability of a ratio being indicative of cancer can be determined.

In various embodiments, the ratio itself as compared to a ratio determined from a normal/control/non-cancerous sample(s) can be used to determine the presence of cancer in patient or subject cfDNA samples. The ratio from a cancer sample will be lower than a ratio from one or more normal samples. Normal samples can be used to establish a threshold ratio, below which a ratio may indicate the presence of cancer.

A classifier can be trained using normal samples and publicly available data sets. Any classifier or machine learning algorithm may be used to analyze the data including, for example, a support vector machine (SVM), linear SVM or kernelized SVM, random forest, elastic net with constrained coefficients, a boosting algorithm, or other classifier.

The Ratio together with protection scores are used as input to the trained classifier. The classifier processes the input ratio. The output from the classifier is a score between 0 and 1, where 0 indicates healthy, no cancer present. A score approaching 1 indicates cancer. The closer the score is to 1, the more it indicates that cancer is present. A threshold for the score can be developed using a set of non-cancerous samples (also referred to as “normal samples”), such that a score greater than the threshold indicates the presence of cancer. The more training data that is used to train the classifier results in a more accurate classifier that can indicate the presence or absence of cancer with greater certainty.

Other fragmentation markers can be analyzed using the methods disclosed herein. Certain regions of the genome are naturally protected from fragmentation. Using genomic data from shallow WGS, one can estimate these protected regions of DNA, where it is expected that fragmentation will not occur. Using these observed regions, we can estimate the likelihood of intact (not fragmented) inserts.

In a patient or subject sample, the method looks to see if the patient or subject has the expected protected regions in their DNA, i.e. the same or similarly protected regions as in normal (non-cancerous) samples/data. When fragmentation is observed in the expected protected regions, it can be indicative of cancer.

The disclosed assay can be designed to amplify certain inserts, for example, in the expected protected regions. Because the assay amplifies only the whole insert, it will not amplify (amplification requires the insert to be intact fragments of the insert. We expect the protected regions to have a greater abundance of whole inserts in healthy (non-cancerous) DNA, as we know that healthy DNA is less fragmented than cancerous DNA. After running the assay, if you count the resulting amplicons and the number is “high” then you can conclude that there was less fragmentation, and therefore less likely to be cancer present. By contrast, if you count the resulting amplicons and the count is low (in a region that was expected to be protected from fragmentation), then you can conclude there was more intense fragmentation in that sample, and therefore is more likely to indicate the presence of cancer.

WGS and shallow WGS data from normal, healthy patients, can be used to create a reference map of the statistical likelihood of fragmented DNA being present in a certain genomic region. The reference map can be created for an entire genome. The reference map from normal healthy patient data is used as a comparison for the patient sample(s) being evaluated for the presence of cancer.

Again, the output from the disclosed assay are amplicon counts. Observing the amplicon in a given region indicates the absence of fragmentation for the DNA insert.

The reference map gives a probability of fragmentation happening in a given region for a normal/healthy patient. Based on this reference, it can be calculated the likelihood of seeing an amplicon count in each area/region of interest. Each amplicon is assigned a score corresponding to the statistical likelihood of that amplicon showing up (referred to herein as “Amplicon protection score”).

Another set of features is generated using what is described herein as unnormalized and normalized protection scores. First, we identify positions corresponding to peaks of cfDNA protection—where the lower the number of fragmentation events in a given position the more protected that position is—in healthy samples via a kernel density estimation (KDE) of the distribution of cfDNA fragment ends positions. As mentioned above, the estimation of the density of fragment ends uses WGS and shallow WGS data from normal, healthy patients. We then determine the average distance between two consecutive peaks that are less than 200 base pairs far from each other. We define a region around a peak as the genomic interval centered around the peak that has a width equal to that average distance. We then restrict our analysis only to the amplicons entirely contained in a region around a given peak. The unnormalized protection score is then defined as the average log-likelihood of observing an unfragmented amplicon, where the average is weighted by the amplicon counts and restricted to counts entirely contained in regions around peaks. The normalized protection score is defined as the average log-likelihood of observing an unfragmented amplicon coming from the short mode minus the average log-likelihood of observing an unfragmented amplicon coming from the long mode, where again the averages are weighted by the amplicon counts and restricted to amplicons contained in regions around peaks. The idea behind normalized protection scores is that the decrease in protection around a peak of protection should be more pronounced in healthy samples when we drift away from the peak compared to that observed with cancerous samples.

A classifier can be trained using normal samples and publicly available data sets. Any classifier or machine learning algorithm may be used to analyze the data including, for example, a support vector machine (SVM), linear SVM or kernelized SVM, random forest, elastic net with constrained coefficients, a boosting algorithm, or another classifier.

The ratio together with protection scores is used as input to the trained classifier. The classifier processes the input ratio. The output from the classifier is a score between 0 and 1, where 0 indicates healthy. A score approaching 1 indicates cancer. The closer the score is to 1, the more it indicates greater certainty that cancer is present. A threshold for the score can be developed using a set of normal samples, such that a score greater than the threshold indicates the presence of cancer. The more training data that is used to train the classifier results in a more accurate classifier that can indicate the presence or absence of cancer with greater certainty.

### Detecting Aneuploidy Using the Instant Methods

Building on the methods disclosed above to detect fragmentation patterns, it is possible to detect aneuploidy with the same data used to detect fragmentation. To achieve this goal, the first step is to cluster amplicon reads into reads coming from short modes and reads coming from long modes as in the previous section. However, instead of taking the ratio between the two mode's counts we consider each mode's counts separately and normalize the counts via dividing each amplicon count by the total number of counts coming from the corresponding mode and the corresponding chromosome arm. After this normalization, we use the following procedure to generate two aneuploidy scores for each chromosome arm. Starting with the short mode, randomly samples are selected within a number of amplicons from each chromosome arm. This number should be the same for every arm. Normalized counts from all selected amplicons of all arms are aggregated. Each column, of a resulting table of data, corresponds to a sample and each row will correspond to an amplicon. Each entry is the normalized counts for the corresponding sample and amplicon. To generate a score for every sample and every arm, a gene set variation analysis (GSVA) is performed where the amplicon counts are viewed as expressions and the chromosome arms as gene pathways. We then repeat the same procedure with the long mode's amplicons. The random sampling of the amplicons is repeated and the average of each of the two scores can be used to gain robustness against batch-effects affecting particular amplicons. This procedure allows to have a specific chromosome arm aneuploidy score for each sample but also an overall aneuploidy classification procedure when the arm specific scores are used as features.

The instant methods work by utilizing repeat regions of the genome and designing two PCR amplicons with an expected unique insert size between the repetitive 5mer and 7mer. The two primer sets will bind to many locations across the genome and create an average amplicon size of about 50 bp and about 70 bp. This bimodal distribution should present differently in normal vs cancer patients.

### Differences in Motifs Distributions

Due to changes in fragmentation patterns and locations of protected regions between cancer and normal samples, the distribution of motifs present should be different between the cancer and normal samples. In our amplicon-based approach, every amplicon, defined as the union of the insert and the primer pairs, represents a particular motif. We can therefore evaluate the motifs profile and/or distribution of a given sample by evaluating the distribution of the motifs associated with the amplicons obtained.

### Motifs Method Description

We start by mapping every fragment/read to the reference genome for every sample. A read is defined as the union of the motifs, including the primer pairs, and the insert. We separate the obtained reads into two categories: long reads, and short reads. Long reads are reads of length corresponding to the long mode (long amplicons) and the short reads to the short mode.

The analysis that we describe is applied independently to each mode. We will therefore describe it for a given fixed mode (short or long)

Focusing for example on the long mode, a sample is represented by a probability distribution over long amplicons. The probability of every long amplicon among long amplicons is simply the proportion of reads coming from the particular amplicon among the long amplicons.

This translates to a probability distribution over strings of letters. More precisely every long amplicon is assigned to a string of letters corresponding to the string obtained from the reference genome when aligning the long read to the reference. Using this sample representation, we would like to measure the similarity between two given samples by measuring the similarity between two probability distributions over strings of fixed length (i.e., long mode in our description).

First, let us define how we compute the similarity between two strings. For this, we test three different similarities. Let us call them the hamming similarity, the spectrum similarity, and the frequency similarity. In the hamming similarity, we represent a k-mer as a k by 4 matrix where each row is filled with zeros except for the position corresponding to the observed letter (A, C, G, or T). For example, if the first letter is C, then the first row is [0100]. Finally, the similarity between two k-mers is nothing but the inner product of their two matrix representations. In the spectrum similarity, we represent the k-mer by a vector of length k\hat{ }3, where every entry corresponds to the frequency of each possible 3-mer substring in the k-mer. The similarity is again the inner product between the 2 vector representations. In the frequency representation, we represent a string by a vector of length 4 where each entry is the proportion of every letter in the k-mer. The similarity is again the inner product between the two representations.

Now that we know how to represent a 6-mer, recall that a sample is represented by a probability distribution over k-mers. The sample probability vector representation is nothing but the expected value (weighted average) of the vector representations of the 6k-mers, where the outcomes are the representations of each k-mer and the probabilities are the probability of every one of those k-mers in the sample. Finally, the similarity/kernel between two different samples is the inner product of the expected value representations of two samples.

Finally, we apply kernel SVM with the three custom-designed kernels as described above. Among the three kernels tested, we observed the best performance using the spectrum representation kernel.

### ROC Curves

FIGS. 15A-15C present ROC curves summarizing the performance on 414 samples of each of the three markers: fragmentation, aneuploidy, and motif distributions. The horizontal axis represents the false positive rate (FP), and the vertical axis represents the true positive rate (TP). In each of the three cases, we performed a 10-fold cross-validation. The dark, solid line represents the average ROC curve over the 10 folds. FIG. 15A represents classification using motifs, FIG. 15B represents fragmentation patterns, and FIG. 15C represents aneuploidy.

In some embodiments, the disclosed method uses three types of features based on the amplicon-based generated data: 1) fragmentation patterns and length; 2) aneuploidy; and 3) motifs distribution.

This disclosure, its aspects and embodiments, are not limited to specific cancers. Accordingly, for example, although particular implementations are disclosed, such implementations may comprise any components, materials, quantities, and/or the like as is known in the art for such methods consistent with the intended operation.

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

We claim:

1. A method of detecting cancer comprising:
providing a human DNA sample from at least one subject suspected of having cancer;
amplifying two or more regions of interest in the human DNA using polymerase chain reaction (PCR) to produce amplified products;
analyzing the amplified products to determine a length distribution of sequences of the amplified products; and
comparing the length distribution of the amplified products with an analogous length distribution of a non-cancerous human DNA sample;
wherein a statically significant difference in distribution of the subject suspected of having cancer versus the distribution of the non-cancerous human DNA sample indicates the likely presence of cancer;
wherein the PCR is performed using a set of PCR primers for each region of interest, the primers comprising a forward primer comprising an about 10-25 base pairs first sequencing primer followed a first 4-8 base pair kmer in the 5′ to 3′ direction and (ii) a reverse primer comprising 4-8 base pair second kmer followed by an about 10-25 bp second sequencing primer in the 3′ to 5′ direction, and
wherein the amplified products comprise a first amplicon with an average length of 35 to 45 base pairs and a second amplicon with an average length of 55 to 65 base pairs between the 5′ to 3′ and 3′ to 5′ primers.

2. The method of claim 1, wherein the first amplicon has an average length of about 40 base pairs and the second amplicon has an average length of about 60 base pairs between the 5′ to 3′ and 3′ to 5′ primers.

3. The method of claim 1, wherein the first amplicon has an average total length of 47 to 52 base pairs and the second amplicon has an average total length of 67 to 72 base pairs.

4. The method of any one of claims 1 to 3, wherein the first amplicon and the second amplicon are produced by two different primers.

5. The method of any one of claims 1 to 4, wherein the first amplicon and the second amplicon are produced by a primer pair or a three primer configuration comprising two forward and one reverse primer.

6. The method of any one of claims 1 to 5, wherein the primers comprise (i) an about 10-25 base pair first sequencing primer followed by 5-7 base pair first kmer in the 5′ to 3′ direction and (ii) a 5-7 base pair second kmer in the 3′ to 5′ direction followed by an about 10-25 bp second sequencing primer.

7. The method of claim 6 wherein one of the first and second kmers comprises 5 base pairs and the other kmer comprises 7 base pairs.

8. The method of any one of claims 1 to 7, wherein the non-cancerous length distribution is obtained from human DNA of one or more non-cancerous subjects.

9. The method of any one of claims 1 to 8, further comprising obtaining an aneuploidy score comprising:
clustering first and second amplicon counts into a short mode and a long mode respectively;
normalizing each count by dividing each amplicon count by the total number of amplicon counts coming from the corresponding mode and the corresponding chromosome arm;
randomly sampling a first number of amplicons from each mode;
generating a first score for every sample and mode within the first number of amplicons and performing a first gene set variation analysis (GSVA);
randomly sampling a second number of amplicons from each mode;
generating a second score for every sample and mode within the second number of amplicons and perform a second gene set variation analysis (GSVA);
averaging the scores from the first GSVA and the second GSVA; and
determining mode specific aneuploidy scores.

10. A method of detecting a cancer in a human patient by analyzing genomic DNA fragmentation, the method comprising:
providing a human DNA sample from the human patient;
amplifying a plurality of regions of interest in the DNA using polymerase chain reaction (PCR) to produce amplified products;
analyzing the amplified products to determine a length distribution of sequences of the amplified products; and
comparing the length distribution of the amplified products with an analogous length distribution of a non-cancerous human DNA sample;
wherein an increase in the ratio of shorter amplified products to longer amplified products in the length distribution of the DNA sample from the human patient compared to that in the non-cancerous human DNA sample indicates increased genomic DNA fragmentation and identifies the cancer.

11. The method of claim 10, wherein
the PCR is performed using a set of PCR primers for each region of interest,
each set of PCR primers comprises a forward primer with a first 4-8 base pair kmer in the 5′ to 3′ direction and a reverse primer with a 4-8 base pair second kmer in the 3′ to 5′ direction,
the first kmer and second kmer in a set of PCR primers are selected to amplify genomic DNA from the human patient resulting in a population of amplicon lengths with a mode characteristic of each region of interest,
the modes characteristic of the plurality of the regions of interest are determined from the amplified products,
and the difference in length between modes is at least 10 bp, at least 15 bp, at least 20 bp, at least 25 bp, or at least 30 bp.

12. The method of claim 11, wherein the plurality of modes comprises a first mode of about 35 bp to about 55 bp and a second mode of about 55 bp to about 75 bp.

13. A method of detecting a cancer in a human patient by analyzing motif distributions, the method comprising:
providing a human DNA sample from the human patient;
amplifying a plurality of regions of interest in the DNA using polymerase chain reaction (PCR) to produce amplified products wherein the amplified products comprise a plurality of motifs;
mapping each motif to a genomic region from the human DNA sample;
determining a probability distribution for each motif to generate a profile of motif distributions for the human DNA sample; and
comparing the profile of motif distributions for the human DNA sample to an analogous profile of motif distributions from a non-cancerous human DNA sample;
wherein significant differences in probability distributions for the human DNA sample and the non-cancerous human DNA sample identifies the cancer.

14. The method of claim 13, wherein analyzing motif distributions comprises analysis with kernel support vector machine (SVM).

15. The method of claim 14, wherein kernel SVM comprises spectrum representation kernel.

16. The method of any one of claims 1 to 15, wherein the human DNA sample and/or the human non-cancerous DNA sample is cell-free DNA.

17. The method of any one of claims 1 to 16, further comprising administering a cancer therapy to the human patient.

18. The method of claim 17, wherein the cancer therapy is surgical resection, chemotherapy, radiation therapy, or a combination thereof.

19. The method of any one of claims 1 to 18, wherein detecting the cancer further comprises aiding in cancer diagnosis; disease monitoring prior to, during, and/or after treatment; minimal residual disease (MRD) detection; or any combination thereof.

20. A method of detecting aneuploidy comprising:
providing a human DNA sample from at least one subject suspected of having cancer;
amplifying two or more regions of interest in the human DNA using polymerase chain reaction (PCR) to produce amplified products;
analyzing the amplified products to determine an amplicon count for each amplified product length;
clustering the first and second amplicon counts into a short mode and a long mode respectively;
normalizing each count by dividing each amplicon count by the total number of amplicon counts and the corresponding chromosome arm;
randomly sampling a first number of amplicons from each mode;
generating a first score for every sample and mode within the first number of amplicons and perform a first gene set variation analysis (GSVA);
randomly sample a second number of amplicons from each mode;
generating a second score for every sample and mode within the second number of amplicons and perform a second gene set variation analysis (GSVA);
averaging the scores from the first GSVA and the second GSVA; and
determining mode specific aneuploidy scores;
wherein the PCR is performed using a set of PCR primers for each region of interest, the primers comprising a forward primer comprising an about 10-25 base pairs first sequencing primer followed a first 4-8 base pair kmer in the 5′ to 3′ direction and (ii) a reverse primer comprising 4-8 base pair second kmer followed by an about 10-25 bp second sequencing primer in the 3′ to 5′ direction, and
wherein the amplified products comprise a first amplicon with an average length of 35 to 45 base pairs and a second amplicon with an average length of 55 to 65 base pairs between the 5′ to 3′ and 3′ to 5′ primers.

21. The method of claim 20, wherein the first amplicon and the second amplicon are produced by two different primers.

22. The method of claim 20, wherein the first amplicon and the second amplicon are produced by one primer.

23. The method of claim 20, wherein the first amplicon has an average total length of 47 to 52 base pairs and the second amplicon has an average total length of 67 to 72 base pairs.

24. The method of claim 20, wherein the first amplicon has an average total length of about 50 base pairs and the second amplicon has an average total length of about 70 base pairs.

25. The method of any one of claims 20 to 24, wherein one of the first and second kmers comprises 5 base pairs and the other kmer comprises 7 base pairs.

26. A method of selecting a primer of the structure SP-kmer wherein SP is sequencing primer and kmer comprises 4-8 base pairs that are commonly positioned immediately to the 5′ or 3′ side of a target sequence in a human DNA sample;
the method comprising:
determining DNA sequences of a plurality of amplicons within target DNA;
determining kmer sequences on the 5′ or 3′ side of the amplicons;
constructing a plurality of test primers;
contacting target DNA with a plurality of test primers and plotting density of fragment count versus fragment length;
selecting a primer that has a single peak of the plot of product count versus product length and has a higher than average density.


SEQUENCE LISTING (60 sequences)

SEQ ID NO: 1 - DNA, 20 bp, Homo sapiens
  source 1..20: mol_type = genomic DNA
aggttaaagccattctcctg

SEQ ID NO: 2 - DNA, 20 bp, Homo sapiens
  source 1..20: mol_type = genomic DNA
tctgtaatcccagctacttg

SEQ ID NO: 3 - DNA, 20 bp, Homo sapiens
  source 1..20: mol_type = genomic DNA
aatggtatgatctcggctca

SEQ ID NO: 4 - DNA, 20 bp, Homo sapiens
  source 1..20: mol_type = genomic DNA
aaatggtatgatctcggctc

SEQ ID NO: 5 - DNA, 20 bp, Homo sapiens
  source 1..20: mol_type = genomic DNA
ttcaaactagagtgcagtgg

SEQ ID NO: 6 - DNA, 20 bp, Homo sapiens
  source 1..20: mol_type = genomic DNA
gaggtaggagaattgcttga

SEQ ID NO: 7 - DNA, 20 bp, Homo sapiens
  source 1..20: mol_type = genomic DNA
aattaattaggcgtggtggc

SEQ ID NO: 8 - DNA, 20 bp, Homo sapiens
  source 1..20: mol_type = genomic DNA
aaaagctgaggcaggagaat

SEQ ID NO: 9 - DNA, 20 bp, Homo sapiens
  source 1..20: mol_type = genomic DNA
ttccaggttcaagcgattct

SEQ ID NO: 10 - DNA, 20 bp, Homo sapiens
  source 1..20: mol_type = genomic DNA
ttaactactcaggaggctga

SEQ ID NO: 11 - DNA, 20 bp, Homo sapiens
  source 1..20: mol_type = genomic DNA
ttttattcaggctggagtgc

SEQ ID NO: 12 - DNA, 20 bp, Homo sapiens
  source 1..20: mol_type = genomic DNA
aagtgtagtggtgtgatctc

SEQ ID NO: 13 - DNA, 20 bp, Homo sapiens
  source 1..20: mol_type = genomic DNA
attttgttgtccaggctgga

SEQ ID NO: 14 - DNA, 24 bp, Homo sapiens
  source 1..24: mol_type = genomic DNA
gctaattcccgggaattttgtagc

SEQ ID NO: 15 - DNA, 22 bp, Homo sapiens
  source 1..22: mol_type = genomic DNA
ccactgcactccaacctaaacg

SEQ ID NO: 16 - DNA, 22 bp, Homo sapiens
  source 1..22: mol_type = genomic DNA
acaggtgtataccaccacaccc

SEQ ID NO: 17 - DNA, 22 bp, Homo sapiens
  source 1..22: mol_type = genomic DNA
ttacaggtatgtgccaccacgc

SEQ ID NO: 18 - DNA, 22 bp, Homo sapiens
  source 1..22: mol_type = genomic DNA
attctcctatctcagcctcccg

SEQ ID NO: 19 - DNA, 22 bp, Homo sapiens
  source 1..22: mol_type = genomic DNA
gcactccagtttgggtgacaga

SEQ ID NO: 20 - DNA, 22 bp, Homo sapiens
  source 1..22: mol_type = genomic DNA
atcacttgagcccaggagttcg

SEQ ID NO: 21 - DNA, 22 bp, Homo sapiens
  source 1..22: mol_type = genomic DNA
actccagtttgggtgacagagc

SEQ ID NO: 22 - DNA, 24 bp, Homo sapiens
  source 1..24: mol_type = genomic DNA
gctaattcccgggattttagtagc

SEQ ID NO: 23 - DNA, 22 bp, Homo sapiens
  source 1..22: mol_type = genomic DNA
ccactgcactccaacctaaacg

SEQ ID NO: 24 - DNA, 22 bp, Homo sapiens
  source 1..22: mol_type = genomic DNA
agcctcccaagtagctgggatt

SEQ ID NO: 25 - DNA, 22 bp, Homo sapiens
  source 1..22: mol_type = genomic DNA
agtagctgggattataggcgcc

SEQ ID NO: 26 - DNA, 22 bp, Homo sapiens
  source 1..22: mol_type = genomic DNA
ctcagcctcccaaataactggg

SEQ ID NO: 27 - DNA, 24 bp, Homo sapiens
  source 1..24: mol_type = genomic DNA
ctgtagtcccagctacttaggagg

SEQ ID NO: 28 - DNA, 24 bp, Homo sapiens
  source 1..24: mol_type = genomic DNA
gctacaaaattcccgggaattagc

SEQ ID NO: 29 - DNA, 22 bp, Homo sapiens
  source 1..22: mol_type = genomic DNA
cgtttaggttggagtgcagtgg

SEQ ID NO: 30 - DNA, 22 bp, Homo sapiens
  source 1..22: mol_type = genomic DNA
gggtgtggtggtatacacctgt

SEQ ID NO: 31 - DNA, 22 bp, Homo sapiens
  source 1..22: mol_type = genomic DNA
gcgtggtggcacatacctgtaa

SEQ ID NO: 32 - DNA, 22 bp, Homo sapiens
  source 1..22: mol_type = genomic DNA
cgggaggctgagataggagaat

SEQ ID NO: 33 - DNA, 22 bp, Homo sapiens
  source 1..22: mol_type = genomic DNA
tctgtcacccaaactggagtgc

SEQ ID NO: 34 - DNA, 22 bp, Homo sapiens
  source 1..22: mol_type = genomic DNA
cgaactcctgggctcaagtgat

SEQ ID NO: 35 - DNA, 22 bp, Homo sapiens
  source 1..22: mol_type = genomic DNA
gctctgtcacccaaactggagt

SEQ ID NO: 36 - DNA, 24 bp, Homo sapiens
  source 1..24: mol_type = genomic DNA
gctactaaaatcccgggaattagc

SEQ ID NO: 37 - DNA, 22 bp, Homo sapiens
  source 1..22: mol_type = genomic DNA
ccactgcactccaacctaaacg

SEQ ID NO: 38 - DNA, 22 bp, Homo sapiens
  source 1..22: mol_type = genomic DNA
aatcccagctacttgggaggct

SEQ ID NO: 39 - DNA, 22 bp, Homo sapiens
  source 1..22: mol_type = genomic DNA
ggcgcctataatcccagctact

SEQ ID NO: 40 - DNA, 22 bp, Homo sapiens
  source 1..22: mol_type = genomic DNA
cccagttatttgggaggctgag

SEQ ID NO: 41 - DNA, 24 bp, Homo sapiens
  source 1..24: mol_type = genomic DNA
cctcctaagtagctgggactacag

SEQ ID NO: 42 - DNA, 24 bp, Homo sapiens
  source 1..24: mol_type = genomic DNA
ctgtagtcccagctacttaggagg

SEQ ID NO: 43 - DNA, 70 bp, Homo sapiens
  source 1..70: mol_type = genomic DNA
aatgatacggcgaccaccgagatctacacnnnnnnnnacactctttccctacacgacgctcttccgatct

SEQ ID NO: 44 - DNA, 66 bp, Homo sapiens
  source 1..66: mol_type = genomic DNA
caagcagaagacggcatacgagatnnnnnnnngtgactggagttcagacgtgtgctcttccgatct

SEQ ID NO: 45 - DNA, 20 bp, Homo sapiens
  source 1..20: mol_type = genomic DNA
aggttaaagccattctcctg

SEQ ID NO: 46 - DNA, 20 bp, Homo sapiens
  source 1..20: mol_type = genomic DNA
tctgtaatcccagctacttg

SEQ ID NO: 47 - DNA, 20 bp, Homo sapiens
  source 1..20: mol_type = genomic DNA
aatggtatgatctcggctca

SEQ ID NO: 48 - DNA, 20 bp, Homo sapiens
  source 1..20: mol_type = genomic DNA
aaatggtatgatctcggctc

SEQ ID NO: 49 - DNA, 20 bp, Homo sapiens
  source 1..20: mol_type = genomic DNA
cgctttcccaaaattagcca

SEQ ID NO: 50 - DNA, 20 bp, Homo sapiens
  source 1..20: mol_type = genomic DNA
ttcaaactagagtgcagtgg

SEQ ID NO: 51 - DNA, 20 bp, Homo sapiens
  source 1..20: mol_type = genomic DNA
gaggtaggagaattgcttga

SEQ ID NO: 52 - DNA, 20 bp, Homo sapiens
  source 1..20: mol_type = genomic DNA
aattaattaggcgtggtggc

SEQ ID NO: 53 - DNA, 20 bp, Homo sapiens
  source 1..20: mol_type = genomic DNA
aaaagctgaggcaggagaat

SEQ ID NO: 54 - DNA, 20 bp, Homo sapiens
  source 1..20: mol_type = genomic DNA
ttccaggttcaagcgattct

SEQ ID NO: 55 - DNA, 20 bp, Homo sapiens
  source 1..20: mol_type = genomic DNA
ttaactactcaggaggctga

SEQ ID NO: 56 - DNA, 20 bp, Homo sapiens
  source 1..20: mol_type = genomic DNA
ttttattcaggctggagtgc

SEQ ID NO: 57 - DNA, 20 bp, Homo sapiens
  source 1..20: mol_type = genomic DNA
aagtgtagtggtgtgatctc

SEQ ID NO: 58 - DNA, 19 bp, Homo sapiens
  source 1..19: mol_type = genomic DNA
attttgttgtccaggctgg

SEQ ID NO: 59 - DNA, 57 bp, Homo sapiens
  source 1..57: mol_type = genomic DNA
cgacgtaaaacgacggccagtnnnnnnnnnnnnnnnnggtgaaaccccgtctctaca

SEQ ID NO: 60 - DNA, 20 bp, Homo sapiens
  source 1..20: mol_type = genomic DNA
ggtgaaaccccgtctctaca